(19) United States
(12) Patent Application Publication
(10) Pub. No.: US 2024/0347144 A1
(43) Pub. Date: Oct. 17, 2024

(54) Artificial Intelligence-based System for Replacing Specific Solvents and Ingredients in Industrial Processes

(71) Applicant: Bioeutectics Corporation, Wilmington, DE (US)

(72) Inventors: Tomás Silicaro, Nordelta (AR); Sergio DavidPasini Cabello, Ayacucho (AR); Maria Romina Canales, Junin Mendoza (AR)

(73) Assignee: Bioeutectics Corporation, Wilmington, DE (US)

(21) Appl. No.: 18/631,100

(22) Filed: Apr. 10, 2024

Related U.S. Application Data
(60) Provisional application No. 63/458,674, filed on Apr. 12, 2023.

(51) Int. Cl. G16C 20/70; G06N 20/20

(52) U.S. Cl. CPC G16C 20/70; G06N 20/20

(57) ABSTRACT

The present invention relates to a system and method for replacing specific solvents and ingredients used in industrial processes with eutectic solvents and mixtures that meet specific characteristics using artificial intelligence. The system is trained and continually updated using experimental formation results obtained in laboratories. The platform is capable of determining whether a completely new system can be formed and predicting some of its physical characteristics. The system is designed to be applied to industrial processes where specific solvents and ingredients are used, and it identifies eutectic solvents that meet or exceed the required characteristics to replace the specific solvent/ingredient. Unlike one process-based approaches, this method does not apply to a specific process, but rather to processes where the specific solvent/ingredient being replaced is used. This present invention provides an effective approach for reducing the use of specific solvents and promoting the use of environmentally-friendly eutectic solvents in industrial processes using artificial intelligence.

This application claims priority under 35 USC 119 (c) to U.S. Provisional Application No. 63/458,674 filed Apr. 12, 2023, the entire contents of which is incorporated herein by reference.

The present document relates to a system and method for replacing specific solvents and ingredients used in industrial processes with eutectic solvents and mixtures that meet specific characteristics using artificial intelligence. The system is trained and continually updated using experimental formation results obtained in laboratories. The platform is capable of determining whether a completely new system can be formed and predicting several of its physical characteristics. The system is designed to be applied to industrial processes where specific solvents/ingredients are used, and it identifies eutectic solvents and mixtures of it that meet or exceed the required characteristics to replace the specific solvent. Unlike one process-based approach, this method docs not apply to a specific process, but rather to processes where the specific solvent/ingredient being replaced is used. This present document provides an effective approach for reducing the use of specific solvents and promoting the use of environmentally-friendly eutectic solvents in industrial processes using artificial intelligence.

### BACKGROUND OF THE INVENTION

The integration of Artificial Intelligence (AI) with other technologies should provide a number of advantages beyond what the technology by itself can provide. Included in these advantages are such things as reduced risk, constant and continuous availability, reduced human error and unbiased decisions, digital computer assistance, greater precision on repetitive tasks, faster decisions, and pattern identification that lead to new and improved outcomes.

One such use of AI can be seen in U.S. Pat. No. 11,1644,478 that attempts to accomplish many of these goals in which AI is used in conjunction with food items. The AI in this patent uses a prediction model that can be trained using features of the source ingredients to match those of the given target food item.

In the chemical field, the implementation of AI should also ideally be designed to accomplish many of these advantages. Because many of the functions in the chemical field are inherently dangerous, the use of AI should be able to reduce risk in that computers can perform some functions that were previously done by individuals, and decisions will be more rationally based to potentially reduce risks. AI also should also be able to reduce risk by scanning known dangers from databases of chemical reactions, compounds and/or solvents. Although AI can be expensive to set-up, once the AI system is in place, costs can be reduced by implementing faster and more rational decisions, and reducing errors that result from erroneous human decisions.

To date, little has been done in connection with the use of AI combined with chemistry, and in particular, deep eutectic solvents. Deep eutectic solvents or DESs are solutions of Lewis or Brønsted acids and bases which form a eutectic mixture. Deep eutectic solvents are highly tunable by varying the structure of the components or by varying the relative ratios of various components in the mixture. Because these are complicated systems that have widely varying properties, they have a wide variety of potential applications, including their use in catalysis, separation techniques, and electrochemical processes. The parent components of deep eutectic solvents tend to engage in complex hydrogen bonding networks or hydrophobic interactions, which means that the mixture tends to have significant freezing point depressions relative to the parent compounds/components in the mixture. Sometimes the individual components in the mixture may be solids at room temperature and atmospheric pressure, but when they are mixed together at room temperature and atmospheric pressure, the mixture may be a liquid that has a severely depressed freezing point (e.g., −80° C.).

NADES are eutectic mixtures formed by a combination of natural compounds with a specific molar ratio. Deep eutectic solvents (DESs) are a newly discovered class of mixtures of solvents that are characterized by significant melting point depressions relative to the corresponding neat constituent components. NADES materials are promising for a plurality of applications as inexpensive solvents that demonstrate a host of tweakable (tunable) physicochemical properties. These solvent systems tend to have unpredictable characteristics because the microscopic mechanisms that govern the structure-property relationships in this class of solvents are generally pretty poorly understood. Complex hydrogen bonding and/or hydrophobic interactions are postulated as the root causes of their melting point depressions and is an attribute that some contend accounts for their physicochemical properties. By adjusting relative amounts of the various components one is able to attain properties that may not be attained by other relative amount compositions even if they contain the same components. However, as NADES related systems are being newly discovered and explored, one must not only understand these supramolecular NADES networks, but it is also imperative to discover their properties by studying the systems as dynamic entities using both simulations and experiments.

The term “eutectic” was first coined in 1884 bp British chemist and physicist Frederick Guthrie. The first generation of eutectic solvents were based on mixtures of quaternary ammonium salts with hydrogen bond donors such as amines and/or carboxylic acids. Natural deep eutectic solvents (NADES) are biologically based deep eutectic solvents which are composed of two or more compounds that are generally plant based primary metabolites, i.e., organic acids, sugars, alcohols, amines and amino acids. Water may also be present as part of the solvent, as a component.

Much of the study of eutectic solvents since Frederick Guthrie coined the term “eutectic” has involved solvent mixtures wherein at least one of the components is a metal based solvent. However, the discharge of metals from these solvent systems has demonstrated many of the drawbacks associated with metal leaching, and its associated health, environmental, and safety related issues. Accordingly, there has been some recent interest in non-metal containing eutectic systems.

Although, deep eutectic solvents/solutions have themselves proved to be useful to substitute for and/or to supplement non eutectic solutions/solvents, it would be useful to have a system that allows for the use of AI in connection with industrial chemical processes, and/or also with the use of deep eutectic solvent systems.

### BRIEF SUMMARY OF THE INVENTION

The present invention relates to a system and method for replacing specific solvents and ingredients used in several industrial processes with eutectic solvents that meet specific characteristics using artificial intelligence. In an embodiment, the system is trained and/or continually updated using experimental formation results obtained in laboratories. In one variation, the platform is capable of determining whether a completely new system can be formed and predicting some, many, and/or all of its physical characteristics. In an embodiment, the system is designed to be applied to industrial processes where specific solvents are used, and it identifies eutectic solvents that meet (or potentially exceed) the required characteristics to replace the specific solvent. Unlike one process-based approaches, in one embodiment, the method may not apply to a specific process, but rather to processes where the specific solvent/ingredients being replaced is used. In an embodiment, the present invention alternatively and/or additionally provides an effective approach for reducing the use of specific solvents and promoting the use of environmentally-friendly eutectic solvents in industrial processes using artificial intelligence.

### DESCRIPTION OF THE SEVERAL VIEWS OF THE DRAWING

FIG. 1 shows a general process of applying AI for predicting new solvents.

FIG. 2 shows a flow diagram with a brief description of applying AI to the prediction of new eutectic systems and their features.

FIG. 3 shows Fingerprints obtention.

### DETAILED DESCRIPTION OF THE INVENTION

The present invention relates to a system and method for replacing specific solvents and ingredients used in industrial processes with eutectic solvents that meet specific characteristics using artificial intelligence. In an embodiment, the system is trained and continually updated using experimental formation results obtained in laboratories. In a variation, the platform is capable of determining whether a completely new system can be formed and predicting some of its physical characteristics. In a variation, the system is designed to be applied to industrial processes where specific solvents are used, and the system can identify eutectic solvents that meet or exceed the required characteristics to replace the specific solvent or ingredient. Unlike one process-based approaches, in an embodiment, the method does not apply to a specific process, but rather to processes where the specific solvent being replaced is used. In a variation, the present invention provides an effective approach for reducing the use of specific solvents and promoting the use of environmentally-friendly eutectic solvents in industrial processes using artificial intelligence.

In an embodiment, the AI system of the present invention can use any of a plurality of learning algorithms. In a variation, the learning algorithm (or prediction models) may be one or more of a linear regression algorithm, a logistic regression algorithm, a decision tree algorithm, SVM algorithm, Naïve Bayes algorithm, KNN algorithm, K-means algorithm, Random Forest algorithm, support-vector machine algorithm, gradient boosting algorithm, DBSCAN algorithm, dimensionally reduction algorithm, gradient boosting algorithm, and/or an AdaBoosting algorithm, or combinations thereof. In a variation, the model may involve supervised learning, unsupervised learning, semi-supervised learning, a deep learning algorithm, reinforcement learning, a regression method, an instance-based method, a decision tree learning method, a Bayesian method, a kernel method, a clustering method, an associated rule learning algorithm, an artificial neural network model, a dimensionality reduction method, an ensemble method, and/or other suitable AI approaches. In an embodiment, the algorithm that is used is a Random Forest algorithm. A Random Forest algorithm generally is used in classification and regression problems. It builds decision trees and makes decisions based upon the class that is selected by most trees.

### EXAMPLE

### Algorithm Types:

Initially, a Random Forest model was used (through the scikit-learn library) based on decision trees. This algorithm was chosen because the initial amount of data was relatively low, and this model tends to be simpler and more efficient than other approaches (such as deep learning approaches) thereby giving good results in most cases. It delivers very good results in cases where an AI model is combined with chemical processes, and specifically in cases where predictions on solvents are mandated. This model was used to perform laboratory validations of predictions, which gave good results. In an embodiment, the model gave 42/52 correct predictions.

Subsequently, as the amount of experimental data increased, a neural network model (deep learning) was evaluated using the TensorFlow library. This algorithm was chosen to continue as it is usually more efficient for more complex tasks but requires a larger amount of data to train, even though its prediction methodology is not very interpretable. Results were compared, and this approach gave even better results (in the same testing set with different data from that used to train the model) than the Random Forest case. The neural network model gave 91% accuracy versus 85% in the case of the Random Forest model.

### Database and Conversion to Parameters Used by the Algorithm:

In an embodiment, the model takes information on the compounds from each mixture through Molecular Fingerprints (vectorized representations generated by cheminformatics tools that allows for virtual screening and mapping chemical space). To do this, the structures of each of the compounds that form the mixture are at first represented in SMILES (Simplified Molecular Input Line Entry System) format (which is the molecular structure in text format to be recognized by chemical structure processing programs). The SMILES data is converted to Fingerprints using the RDkit program. The latter consists of a vector that represents the absence (represented by 0) or presence (corresponding to 1 in the vector) of each of the substructures that make up each molecule.

To represent a mixture in the training and testing data of the model, the corresponding fingerprints for each of the molecules that make it up are multiplied by their percentages and subsequently added, in order to reduce bias due to the order of the components, obtaining a dataset that is scaled between 0 and 1. The compounds are thus weighted using their respective percentages when present in a mixture.

The columns (features) for training the model are weighted the same for different predictions (stability and/or physical properties).

The database contains about five thousand compound combinations. In addition to information on the stability of the eutectic mixture, there is information on various physicochemical properties of the eutectic mixtures, including but not limited to properties such as pH, viscosity(y)(ies), density, conductivity, polarity, refractive index, color, odor, antioxidant capacity, and antimicrobial capacity.

For the formation prediction model (which must predict whether a mixture is stable or not), the problem of an imbalance in positive and negative samples was encountered, with the first class (i.e., positive) being overrepresented. In practice and in this embodiment, this issue is translated into a vast majority of predictions made with new combinations being positive for stability.

To address this problem, it was decided to use an unsupervised learning approach. At first, a base model that only contains experimental and bibliographic data, along with compound combinations that are extremely likely to be unstable (determined by advanced knowledge in chemistry about eutectic solvents), was generated. This model was used to classify data coming from random combinations of compounds. Mixtures labeled as negative for stability are added to the final dataset, in order to expand the universe of negatives, which is used to train the definitive classifier. The amount of augmented data was defined according to the accuracy on a test set that contains only experimentally verified data (after the hyperparameter optimization made using the validation set accuracy). By making these modifications, the problem of overrepresented positive weighting was greatly reduced, with approximately 5% of random mixtures classified as positive. The results require experimental verification as to whether any of the 5% positives correspond to true negatives.

For the physical properties prediction models, as there is less experimental data available (the data set is reduced to the combinations that form stable mixtures), it was decided to expand the database with predictions calculated using mixture design predictions. With a small set of experimental data, the mathematical formula is adjusted depending on the components and their respective molar ratios that make up the mixture, and a prediction of the properties is made by modifying those coefficients.

In turn, when the respective compounds in mixtures have a wide variation in their respective properties, such as viscosity, transformations were made using a simplified scale at predefined intervals. To limit the number of responses and the operation of the algorithm, the compounds were classified into groups of data corresponding to ranges. This was performed for many of the physicochemical properties, including but not limited to the pH.

Furthermore, to obtain possible new combinations and compounds capable of forming new stable mixtures, natural compound databases such as Pubchem and Lotus were used. From these databases, compounds with a certain degree of similarity to those known to form eutectic mixtures (by the Tanimoto Similarity Index) were evaluated. The Tanimoto Similarity Index compares Fingerprints of two molecules according to their shared characteristics, giving a value between 0 and 1 according to their similarity. Those with a Tanimoto Similarity Index value of higher than 0.6 were used.

### Training

To train the model, two subsets were separated from the initial dataset: a validation set (external data to the training set to optimize the model) with 100 positives and 100 negatives, and a testing set (representing additional unknown data for the model, to verify that a similar accuracy to the validation set is achieved) with 50 positive data and 50 negative data. All remaining data is used for training, i.e., the data with which the model adjusts its parameters.

There are certain parameters of the models that are fixed throughout the training process, such as the tree size in Random Forest and the learning rate in Neural Networks, which require trial and error to obtain optimal values. For this, the Optuna library was used, which performs a heuristic search and obtains the best values in terms of accuracy in the validation set.

The metric used that obtained the best parameters in stability prediction models was the mean classification error (=correctly classified data/total data), while for physical property predictors, the mean squared error test was used.

In this way, models were generated with appropriate parameters that minimize the error in predictions (both for stability and physicochemical property prediction).

The model ultimately works from the fingerprint columns corresponding to a combination of compounds multiplied by their molar ratios, and the model returns a value between 0 and 1 for each case, with values greater than 0.5 corresponding to combinations that are predicted to be stable. In the case of a physical property prediction, the result is a value corresponding to the prediction of that property, or to one of the possible intervals of that value for simplified approximations.

### Operation Description:

To make predictions, different approaches were proposed. One of the approaches involves starting from a list of components available in the laboratory. The components are mixed and randomized molar ratios are used. In this embodiment, a large number of examples can be produced. Predictions are then made for these various combinations. The model assigns a value between 0 and 1, and the higher the value (i.e., the closer to 1), the more reliable the eutectic mixture. Using this methodology, it is also possible to obtain values for certain physical properties.

In a variation, a second approach involves inputting a new compound into the application and searching for similar compounds that form stable mixtures. The most similar compounds are replaced by this new compound in their respective mixtures, and stability is predicted by using the new replacement component and its similarity to the compounds the new compound replaces.

The last approach consists in using physicochemical characteristics as input data (whether or not naming any component of the eutectic mixture) so that the AI can return possible NADES (natural deep eutectic solvents) that meet these characteristics. In this case, in the first place multiple predictions are made (for stability and properties) and saved in a database. Finally Nearest Neighbor searches are performed to obtain the mixtures whose predicted physicochemical features are as close as possible to the desired ones.

In an embodiment, several approaches or modes can be used:

1. Randomized Combinations: This approach generates randomized combinations of components and molar ratios, and predicts their stability as eutectic mixtures. The model returns a value between 0 and 1, allowing for the ranking of the most reliable eutectic mixtures. Additionally, this approach can predict certain physical properties.
2. Similar Compound Replacement: In this mode, a new compound is inputted, and the application searches for similar compounds that form stable eutectic mixtures. The most similar compounds are then replaced by the new compound in their respective mixtures, and the stability of the modified mixture is predicted.
3. Physicochemical Properties: In this mode, the application is given specific physicochemical properties (with or without mentioning any specific component of the eutectic mixture), and it returns the possible NADES that meet those characteristics/properties. In a variation, this approach can be used with the replacement of solvents approach.

In a variation, one training has occurred, validation of those random combinations can be accomplished.

### Validation

In an embodiment, and as an experiment, 52 random combinations were made from a list of compounds. The stability prediction of these combinations was made with the model, with 17 of them predicted as positive and 35 predicted as negative for formation. These combinations were then evaluated in the laboratory, and for 8 out of the 17 positive, the prediction was correct. In the case of the 35 negative predictions, only 2 resulted in stable mixtures. Accordingly, overall, 41 out of 52 predictions (approximately 79%) were correct (8+33=41), with the majority of true positives (8 out of 10) being in the subset of positive predictions. Thus, by evaluating only the true positives (i.e., disregarding positives that were not true) and the negatives, 41 of 45 combinations gave accurate results.

In an embodiment, the combinations gave accuracy levels that were above about 75%, or above about 80%, or above about 85%, or above about 90%.

Further evaluation remains to be performed for formation using the more complex neural network approach. Moreover, further evaluation remains to be performed for physical-chemical property prediction with an increased data matrix based on equations. The preliminary results are promising relative to previous approaches (such as using the Random Forest algorithm methodology). This approach yields better results (in the same testing set with different data from those used to train the model) relative to the Random Forest case (91% accuracy for the complex neural network approach vs 85% for the Random Forest approach).

As a practical example of solvent replacement in the industry, we can mention the extraction of Lycopene. Lycopene is an antioxidant found in tomatoes that is valued for its properties in combating degenerative diseases. Traditionally, lycopene extraction has been carried out using solvents such as hexane and ethyl acetate, and more recently, through the use of supercritical CO2, which entails high energy consumption. However, through the use of artificial intelligence and considering the physicochemical properties of solvents, we have identified a natural eutectic solvent substitute with similar properties that has demonstrated the ability to selectively extract lycopene from tomatoes. This advance may represent a more sustainable and economical alternative for lycopene extraction in the future.

Examples of stability predictions using the AI are presented in tables 1 and 2. With these results, the algorithm is re-trained in order to learn and increase accuracy.

TABLE 1
Examples of successful predictions and lab tests.

| Compound | Compound | Compound | | AI | Lab |
|---|---|---|---|---|---|
| A | B | C | Ratio | prediction | Test |
| glycerol | eucalyptol | oleic acid | 1:2:1 | Stable | Stable |
| glycerol | lactic acid | eucalyptol | 1:2:1 | Stable | Stable |
| glycerol | thymol | camphor | 1:2:1 | Unstable | Unstable |
| glycerol | borneol | camphor | 1:2:1 | Unstable | Unstable |

TABLE 2
Examples of failed predictions and lab tests.

| Compound | Compound | Compound | | AI | Lab |
|---|---|---|---|---|---|
| A | B | C | Ratio | prediction | Test |
| menthol | oleic acid | water | 1:1:1 | Stable | Unstable |
| menthol | lactic acid | water | 1:1:1 | Stable | Unstable |
| menthol | thymol | water | 1:1:1 | Stable | Unstable |

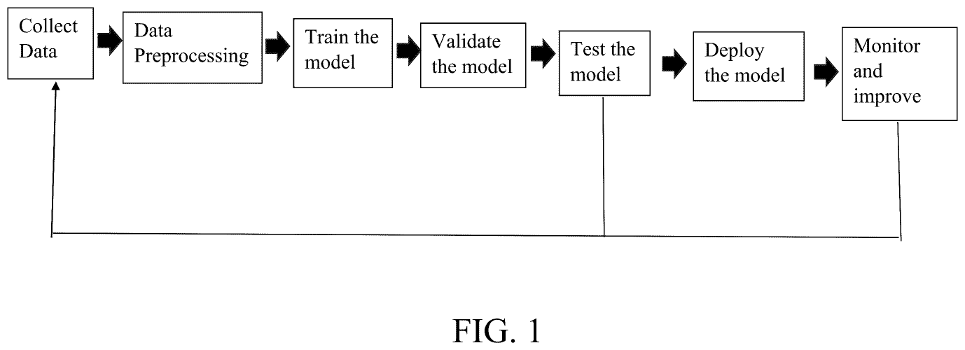

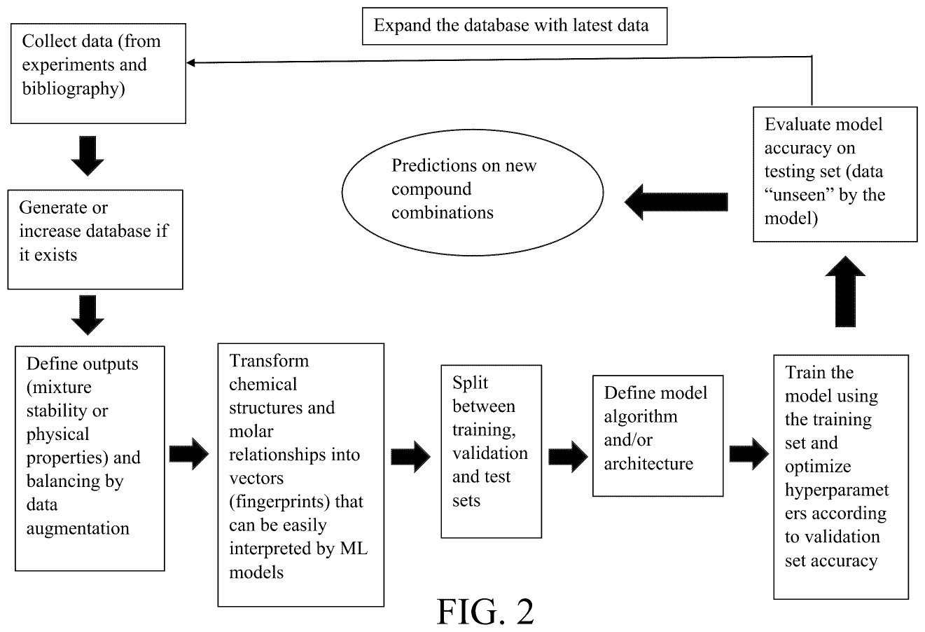

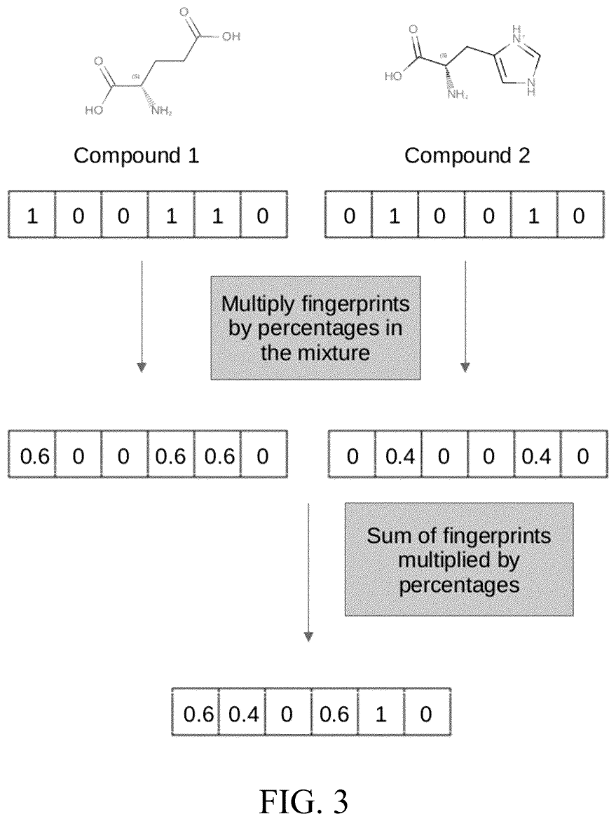

FIG. 1 shows a generalized process of how one can use AI for any purposed including obtaining an idealized solvent system (including validating, testing and deploying the model and then subsequently improving the model). FIG. 2 shows a more specific process of AI that is the process of selecting the idealized solvent system. Note that in this figure, the testing of the solvent system (evaluating the model) generates new data that allows the solvent systems to be continuously improved. FIG. 3 shows the use of the Fingerprints data and the multiplying of the vectors by the respective percentages to arrive at the data that is used in the AI system.

In an embodiment, the present invention relates to a system for identifying ideal solvents or ingredients to use in an industrial process, the system comprising combining eutectic solvents with artificial intelligence. In a variation, the ideal solvent or ingredient is a mixture of two or more solvents/products. In a variation, the ideal solvent is a mixture of three or more solvents. In a variation, the ideal solvents further comprise water.

In an embodiment, the artificial intelligence uses an algorithm, said algorithm comprising one or more members selected from the group consisting of a linear regression algorithm, a logistic regression algorithm, a decision tree algorithm, a SVM algorithm, a Naïve Bayes algorithm, a KNN algorithm, a K-means algorithm, a Random Forest algorithm, a complex neural network algorithm, a support-vector machine algorithm, a gradient boosting algorithm, a DBSCAN algorithm, a dimensionally reduction algorithm, a gradient boosting algorithm, an AdaBoosting algorithm, and combinations thereof.

In a variation, the algorithm is a Random Forest algorithm or a complex neural network algorithm. In a variation, the identifying ideal solvents comprises replacing specific solvents used in an industrial process with eutectic solvents. In a variation, the identifying ideal solvents comprises identifying one or more physicochemical properties. In a variation, the one or more physicochemical properties comprise one or more members selected from the group consisting of pH, viscosity, density, polarity, refractive index, color, odor, antioxidant capacity, and antimicrobial capacity.

In an embodiment of the system, the accuracy for predicting the ideal solvents is at least about 80%, or at least about 85%, or at least about 90%.

In an embodiment, the ideal solvent is a mixture of two or more solvents, wherein the ideal solvent further comprise water, and wherein the artificial intelligence uses an algorithm, said algorithm comprising one or more members selected from the group consisting of a linear regression algorithm, a logistic regression algorithm, a decision tree algorithm, a SVM algorithm, a Naïve Bayes algorithm, a KNN algorithm, a K-means algorithm, a Random Forest algorithm, a complex neural network algorithm, a support-vector machine algorithm, a gradient boosting algorithm, a DBSCAN algorithm, a dimensionally reduction algorithm, a gradient boosting algorithm, an AdaBoosting algorithm, and combinations thereof.

In an embodiment, the present invention relates to a method of identifying an ideal solvent mix for use in an industrial process, the method comprising: inputting data on a plurality of solvents into a computer designed to run an artificial intelligence algorithm wherein the computer comprises the artificial intelligence algorithm, wherein the artificial intelligence algorithm is able to process the data to output useful information on the solvent mix; running the algorithm to generate the useful information; and evaluating the useful information to identify the ideal solvent mix.

In an embodiment, the ideal solvent mix comprises at least one eutectic solvent.

In a variation, the artificial intelligence algorithm comprises one or more members selected from the group consisting of a linear regression algorithm, a logistic regression algorithm, a decision tree algorithm, a SVM algorithm, a Naïve Bayes algorithm, a KNN algorithm, a K-means algorithm, a Random Forest algorithm, a complex neural network algorithm, a support-vector machine algorithm, a gradient boosting algorithm, a DBSCAN algorithm, a dimensionally reduction algorithm, a gradient boosting algorithm, an AdaBoosting algorithm, and combinations thereof. In a variation, the algorithm comprises a Random Forest algorithm or a complex neural network algorithm.

In a variation of the method, the data comprises one or more physicochemical properties selected from the group consisting of pH, viscosity, conductivity, density, polarity, refractive index, color, odor, antioxidant capacity, and antimicrobial capacity.

In a variation, the method further comprises mixing the solvents and randomizing molar ratios of the solvents. In a variation, the molar ratios are not randomized.

In a variation, the accuracy of the method of identifying the ideal solvent mix is at least about 80%. In a variation, the method comprises a training step and a validation step.

In a variation, the validation step further comprises performing experiments to determine the ideal solvent mix.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A system for identifying ideal solvents to use in an industrial process, the system comprising combining eutectic solvents with artificial intelligence.

2. The system of claim 1, wherein the ideal solvent/ingredient is a mixture of two or more components.

3. The system of claim 2, wherein the ideal solvent further comprise water as a component.

4. The system of claim 1, wherein the artificial intelligence uses an algorithm, said algorithm comprising one or more members selected from the group consisting of a linear regression algorithm, a logistic regression algorithm, a decision tree algorithm, a SVM algorithm, a Naïve Bayes algorithm, a KNN algorithm, a K-means algorithm, a Random Forest algorithm, a complex neural network algorithm, a support-vector machine algorithm, a gradient boosting algorithm, a DBSCAN algorithm, a dimensionally reduction algorithm, a gradient boosting algorithm, an AdaBoosting algorithm, and combinations thereof.

5. The system of claim 4, wherein the algorithm is a Random Forest algorithm or a complex neural network algorithm.

6. The system of claim 1, wherein the identifying ideal solvents comprises replacing specific solvents or ingredients used in an industrial process with eutectic solvents.

7. The system of claim 1, wherein the identifying ideal solvents comprises identifying one or more physicochemical properties.

8. The system of claim 7, wherein the one or more physicochemical properties comprise one or more members selected from the group consisting of pH, viscosity, density, conductivity, polarity, refractive index, color, odor, antioxidant capacity, and antimicrobial capacity.

9. The system of claim 5, wherein an accuracy for predicting the ideal solvents is at least about 80%.

10. The system of claim 9, wherein the accuracy for predicting the ideal solvents is at least about 90%.

11. The system of claim 1, wherein the ideal solvents is a mixture of two or more solvents, wherein the ideal solvents further comprise water as component, and wherein the artificial intelligence uses an algorithm, said algorithm comprising one or more members selected from the group consisting of a linear regression algorithm, a logistic regression algorithm, a decision tree algorithm, a SVM algorithm, a Naïve Bayes algorithm, a KNN algorithm, a K-means algorithm, a Random Forest algorithm, a complex neural network algorithm, a support-vector machine algorithm, a gradient boosting algorithm, a DBSCAN algorithm, a dimensionally reduction algorithm, a gradient boosting algorithm, an AdaBoosting algorithm, and combinations thereof.

12. A method of identifying an ideal solvent mix for use in an industrial process, the method comprising:
inputting data on a plurality of solvents into a computer designed to run an artificial intelligence algorithm wherein the computer comprises the artificial intelligence algorithm, wherein the artificial intelligence algorithm is able to process the data to output useful information on the component mix (eutectic solvent);
running the algorithm to generate the useful information; and
evaluating the useful information to identify the ideal solvent mix.

13. The method of claim 12, wherein the ideal solvent mix comprises at least one eutectic solvent.

14. The method of claim 13, wherein the artificial intelligence algorithm comprises one or more members selected from the group consisting of a linear regression algorithm, a logistic regression algorithm, a decision tree algorithm, a SVM algorithm, a Naïve Bayes algorithm, a KNN algorithm, a K-means algorithm, a Random Forest algorithm, a complex neural network algorithm, a support-vector machine algorithm, a gradient boosting algorithm, a DBSCAN algorithm, a dimensionally reduction algorithm, a gradient boosting algorithm, an AdaBoosting algorithm, and combinations thereof.

15. The method of claim 14, wherein the algorithm comprises a Random Forest algorithm or a complex neural network algorithm.

16. The method of claim 12, wherein the data comprises one or more physicochemical properties selected from the group consisting of pH, viscosity, conductivity, density, polarity, refractive index, color, odor, antioxidant capacity, and antimicrobial capacity.

17. The method of claim 12, wherein the method further comprises mixing the solvents and randomizing molar ratios of the solvents.

18. The method of claim 12, wherein an accuracy of the method of identifying the ideal solvent mix is at least about 80%.

19. The method of claim 12, wherein the method comprising a training step and a validation step.

20. The method of claim 19, wherein the validation step further comprises performing experiments to determine the ideal solvent mix.
